(19) United States
(12) Patent Application Publication
(10) Pub. No.: US 2014/0242669 A1
(43) Pub. Date: Aug. 28, 2014

(54) PRODUCTION OF INFECTIOUS RNA VIRUSES IN YEAST

(71) Applicant: YCINE LLC, Glenwood, MD (US)

(72) Inventors: Arun K. Dhar, Sykesville, MD (US); Daniel Bednarik, Highland, MD (US)

(21) Appl. No.: 14/239,194

(22) PCT Filed: Aug. 20, 2012
(86) PCT No.: PCT/US12/51569

§ 371 Date: May 6, 2014

Related U.S. Application Data
(60) Provisional application No. 61/524,854, filed on Aug. 18, 2011.

(51) Int. Cl. C12N 15/86; C12N 7/00

(52) U.S. Cl. CPC C12N 15/86; C12N 7/00
USPC 435/236; 435/320.1

(57) ABSTRACT

The method described herein provides a novel platform utilizing yeast as a biological non-host system to express and assemble whole viruses for use as attenuated or killed vaccines.

### CROSS REFERENCE TO RELATED APPLICATION

This application claims priority to U.S. Provisional Application No.: 61/524,854 filed in the United States Patent and Trademark Office on Aug. 18, 2012, the content of which is hereby incorporated by reference herein for all purposes

### BACKGROUND OF THE INVENTION

1. Field of the Invention

The present invention relates to the Production of Infectious RNA Viruses in Yeast.

2. Related Art

It has been very difficult to produce an effective vaccine against a wide array of diseases such as viral gastroenteritis caused by Norwalk virus (NV), hepatitis caused by hepatitis C virus (HCV), dengue fever caused by dengue virus (DENV), West Nile fever caused by the West Nile virus (WNV), respiratory illness caused by respiratory syncytial virus (RSV) of human and viral hemorrhagic septicemia virus (VHSV), a deadly infectious fish virus. The use of an inactivated wild type virus is generally the most effective way of vaccinating against viral diseases a pre-requisite of which is to grow the vaccine virus in large quantity. However, there are many viruses such as NV, HCV that are either very difficult to grow or a robust cell culture method is non-existing.

### SUMMARY OF THE INVENTION

The invention describes a simple yet novel way of growing viruses in a fermentor using a yeast as a biofactory for producing an infectious RNA virus such NV, HCV, DENV, WNV, RSV, VHSV for vaccine purpose. For an RNA virus containing a single-stranded positive-sense mono-segmented genome such as NV, HCV, DENV and WNV, the method involves cloning a complimentary DNA copy of the viral genome in a yeast expression vector. Subsequently, the plasmid DNA containing the virus genome is used to transform yeast. Upon transformation of yeast, the promoter in the yeast expression vector will drive the expression of the viral genome. The viral RNA is then translated to make viral-encoded protein including the capsid proteins which undergoes proteolytic cleavage to make mature capsid protein. The mature capsid proteins subsequently encapsilate the viral RNA in the yeast cell to form a full-assembled virus.

For an RNA virus containing a single-stranded negative-sense mono-segmented genome such as RSV, the method involves cloning a complimentary DNA copy of the viral genome under the control of one of the two promoters in a yeast expression vector containing dual promoters. The complimentary DNA copy of the viral genome in this case contains a T3 or T7 promoter sequence at the 3′-end of the genome. A T3 or T7 polymerase gene is then cloned under the control of a second promoter in the yeast expression vector. Recombinant plasmid carrying the viral genome and the gene for T3 or T7 polymerase is then used to transform yeast.

Upon transformation of yeast, the promoter upstream of the viral genome will generate positive-sense viral genome which is translated to make viral-encoded protein including the capsid proteins. The second promoter located upstream of the T3 or T7 polymerase gene tanscribes the corresponding gene which is translated to make T3 or T7 polymerase protein. The T3 or T7 polymerase use the +ve-sense viral genome as a template to make −ve-sense RNA. The −ve-sense viral RNA is then packaged by the capsid protein to make a full-assembled virus. In this way, both positive- and negative-sense mono-segmented RNA virus can be produced in large quantities in yeast that can be purified to homogeneity, and inactivated to generate a vaccine or used as a whole cell vaccine without the need to purification for the prevention of the disease.

If the viral genome cloned into the yeast expression vector represents an attenuated form of the virus, the attenuated virus can be purified to homogeneity from yeast and used as a vaccine without the need to inactivating the virus prior to vaccination. Alternatively, the recombinant yeast expressing the attenuated vaccine virus can be used as a whole cell vaccine. In short, the invention describes a novel way to producing a human, and animal viruses, and the uses thereof, in a yeast cell.

### DETAILED DESCRIPTION OF THE INVENTION

### I. Cloning of a Positive-Sense, Single-Stranded, Linear Non-Segmented RNA Virus

Positive-sense, single-stranded, linear non-segmented RNA viruses are classified in the Families, Leviviridae (e.g. Enterobacteria phage MS2 infecting Escherichia coli), Narnaviridae (e.g. Cryptonectria mitovirus 1 infecting chestnut blight fungus Cryptonectria parasitica), Picrona viridae (e.g. Poliovirus, Human rhinovirus, Encephalomyocarditis virus, Foot-and-mouth disease virus, Hepatitis A virus, Equine rhinitis B virus, Aichi virus, Porcine teschovirus, Infectious flacherie virus), Dicistroviridae, Marnaviridae, Sequiviridae, Comoviridae, Potyviridae, Calciviridae (e.g. Rabbit hemorrhagic virus belonging to the genus Largovirus, Norwalk virus belonging to the genus Norovirus, Sapporo virus belonging to the genus Sapovirus and Vesicular exanthema of swine virus belonging to the genus Vesivirus), Astroviridae, Nodaviridae, Tetraviridae infecting Lepidopteran insects, Luteoviridae, Tombusviridae, families belonging to the Order Nidovirales (e.g. Families Coronaviridae, Arteriviridae and Roniviridae), Flaviviridae (e.g. Yellow fever virus in the genus Flavivirus, Bovine viral diarrhea virus 1 in the genus Pestivirus, and Hepatitis C virus in the genus Hepacivirus), genera such as Alphavirus, Rubivirus and Tobamovirus belonging to the family Togaviridae, Tymoviridae, Closteroviridae, and Flexiviridae.

In the following section the invention is described in detail using a series of examples with Norwalk virus. Norwalk virus (NV) is the type species of the genus Norovirus and is the major cause of non-bacterial gastroenteritis worldwide. The virus affects people of all ages and is transmitted by fecally contaminated food or water, by person-to-person contact and via aerosol containing the virus and subsequent contamination of surfaces (Norovirus: Technical Fact Sheet“. National Center for Infectious Diseases, CDC. http://www.cdc.gov/ncidod/dvrd/revb/gastro/norovirus-factsheet.htm; Goodgame R (2006)).

NV is small (27 nm to 35 nm in diameter), round, non-enveloped, and with an amorphous surface structure. The virion contains one single-stranded, positive-sense, 7.7-kb RNA genome which is polyadenylated at the 3′-end and contains a genome-linked protein (VPg) at the 5′ end. Genome sequence analysis has revealed that there are three open reading frames (ORFs). ORF 1 encodes a polyprotein that is processed into nonstructural proteins required for virus replication and shows similarity to picornavirus 2C helicase, 3C protease, and 3D RNA-dependent RNA polymerase. ORF 2 encodes the viral capsid protein, and ORF 3 encodes a small basic protein with an unknown function. NV also produces a 2.3-kb sub-genomic RNA containing ORFs 2 and 3, each of them having a strong AUG initiation codon, suggesting that they may be expressed independently (Jiang 1993)

### EXAMPLE 1

Cloning of a full-length genome of NV in a pCR®-XL-TOPO® vector.

The entire genome of NV can be amplified by RT-PCR using virus-specific primers and cloned into a pCR®-XL-TOPO® vector following manufacturer's recommendations (Invitrogen Inc., Carlsbad, Calif.). Alternatively, the entire genome of NV can be custom synthesized and cloned into a plasmid vector, pUC57 (GenScript USA Inc., 860 Centennial Avenue, Piscataway, N.J. 08854). Upon cloning, the recombinant clones are verified by sequencing before taking for sub-cloning.

### EXAMPLE 2

Sub-cloning of NV in a yeast expression vector and western blot analysis using anti-NV antibody.

Upon successful cloning and sequence validation, the full-length genome of NV may be digested using unique restriction enzyme(s) and cloned into the homologous sites in a yeast expression vector pESC-ura (Stratagene, San Diego, Calif.) following manufacturer's protocol. Alternatively, the entire genome of NV can be amplified by PCR from the plasmid DNA (from Example 1), and cloned into pESC-ura (Stratagene, San Diego, Calif.) by overlapping PCR. The plasmid DNA is then isolated from the recombinant clones and sequenced. The recombinant plasmid is then used to transform yeast (Saccharomyces cerevisiae strain YPH501, Stratagene, La Jolla, Calif,) using a published protocol (http://www. genomics.agilent.com/files/Manual/217451.pdf (Agilent Technologies, Santa Clara, Calif.). After transformation, the transformants are plated on SG-dropout media and grown at 30° C. for two days before taking for western blot analysis.

The western blotting of NV using NV-specific monoclonal antibody is performed following a published protocol (Allnutt et al., 2007). Recombinant yeast (Saccharomyces cerivisiae strain YH501; Stratagene, La Jolla, Calif.) clones containing NV genome are grown in autotrophic SG-ura medium containing galactose, yeast extract without amino acids, and amino acid dropout mixture (all amino acids plus adenine, no uracil) at 30° C. for 4 days. Cells were collected by centrifugation, and crude protein extracts were prepared using Y-PER yeast breaking buffer (Pierce Biotechnology, Rockford, Ill.). An aliquot of total protein is then electrophoresed in a 12% SDS-polyacrylamide gel (BioRad, Richmond, Calif.) and transferred to PVDF membrane by electroblotting. The membrane is then probed with Mouse anti-Norovirus GI.1 Monoclonal Antibody (Abcam Inc., Cambridge, Mass.) and detected with Goat-anti-mouse polyclonal antibody conjugated to HRP (Thermo Fisher Scientific, Rockford, Ill.). Detection is carried out using a ECL Plus Western Blotting Reagent Pack (GE Healthcare, Piscataway, N.J.).

### EXAMPLE 3

Purification of recombinant NV.

Recombinant NV is purified following essentially the same protocols as described for the purification of virus-like particles from recombinant yeast (Allnutt et al., 2007). Briefly, recombinant yeast expressing NV is grown in autotrophic SG-ura medium as described above at 30° C. for 4 days. Cells are then collected by centrifugation, treated with β-glucoronidase to prepare the protoplast following a published protocol (Pannunzio et al., 2004) before storing at −20° C. Protoplasted cells are then lysed by three freeze thaw (3 cycles), and then sonicated by 60 second pulses with 20 second intervals (5 cycles). The sample is then centrifuged at 10,000 rpm for 20 minutes at 4° C. The supernatant is layered over 15-60% (w/v) sucrose gradient and centrifuged at 28,000 rpm for 3 hours at 4° C. in a swinging bucket rotor (Beckman SW20). Multiple fractions are collected throughout the gradient and western blot analysis is performed using anti-NV antibody to determining which fraction(s) contains highest level of the virus. Those fraction(s) are then diluted to 1:10 volume using TN buffer (50 mM Tris and 100 mM NaCl, pH 8.0) and is centrifuged at 28,000 rpm for 2 hours at 4° C. in a swinging bucket rotor (Beckman SW20). The pellet is collected and suspended in TN buffer before using for transmission electron microscopy (TEM).

### EXAMPLE 4

Characterization of NV purified from yeast by transmission electron microscopy, SDS PAGE, and glycosylation pattern.

Transmission electron microscopy (TEM) is performed to determine the morphology and size of the recombinant NV expressed in yeast following to a previously published protocol (Dykstra, 1992). An aliquot of purified NV produced in yeast as described in the Example 3 is boiled in a denaturing buffer containing 0.5% sodium dodecyl sulfate (SDS) and 1% β-mercaptoethanol for 10 minutes, and subjected to SDS polyacrylamide gel electrophoresis (SDS-PAGE) on 12% gels. The proteins from the gel are transferred to nylon membrane and the membrane is probed with anti-NV antibody to demonstrate the reactivity of the purified NV to homologous antibody, as described in Example 2.

In order to determine the glycosylation pattern of the yeast expressed NV, purified virions are pre-treated with 10 U endoglycosidase (Endo) H (New England Biolabs, Beverly, Mass.) per microgram of protein at 37° C. for 3 hours in a buffer containing 50 mmol/L sodium citrate at pH 5.5 prior to denaturation and separation using SDS-PAGE to remove any glycosylation from the viral coat proteins. Untreated purified wild-type NV sample serves as a control. The comparison of NV peptide profiles upon treatment of endoglycosidase vs. control sample is used to determine the extent of glycosylation of the yeast expressed NV relative to that of the native NV. Comparison of deglycosylated control NV proteins with deglycosylated NV proteins produced by the instant invention using SDS-PAGE establishes that the NV proteins produced using the instant invention are processed accurately (i.e., to the same molecular weight as in the native NV).

### EXAMPLE 5

Establishing the infectivity of NV isolated from recombinant yeast.

The ability of the NV produced by the instant invention to infect mammalian cells is demonstrated by the ability of the purified virions produced in Example 3 to replicate and propagate in a human hepatoma cell line (Huh-7), CaCo-2 human intestinal cells following a published protocol (Guix et al., 2007). The viral loads in the infected cells are measured by real-time quantitative RT-PCR following a published protocol for quantifying hepatis A virus (Brooks et al., 2005).

### EXAMPLE 6

Establishing the antigenicity of NV isolated from Example 3.

In order to determine the antigenicity of NV purified from recombinant yeast, gnotobiotic (Gn) pigs are injected with NV and the antibody response is measured in injected pigs and compared to sham injected control. Gnotobiotic (Gn) pig is a more promising model of human noroviruses than non-human primates. In a neonatal Gn pig, human norovirus infection causes diarrhea, virus shedding, seroconversion, immuno-cytopathic change in the intestinal sections, and transient viremia (Tan et al., 2010). This is likely due to the gastrointestinal anatomy, physiology, immune system, and the presence of HBGAs, such as the A and H antigens on mucosal surfaces of pigs are similar to human.

### II. Cloning of a Negative-Sense, Single-Stranded, Linear Non-Segmented RNA Virus

Negative- sense, single-stranded, linear non-segmented RNA viruses are classified in the Order Mononegavirales, family Bornaviridae (Genus: Bornavirus), family Rhabdoviridae (Genus: Vesiculovirus, Lyssavirus, Ephemerovirus, Novirhabdovirus, Cytorhabdovirus, Nucleorhabdovirus), family Filoviridae (Genus: Marburgvirus and Ebola virus), and family Paramyxoviridae (Genus: Rubalavirus, Avulavirus, Respirovirus, Henipavirus, Morbillivirus, Pneumovirus, and Metapneumovirus). Viruses belonging to the order Mononegavirales infect plants, invertebrates and vertebrates, cause a wide range of diseases and sometimes cause fatal infection. For example, Rabies virus (RABV), the type species in the genus Lyssavirus, is the etiological agent of rabies encephalitis in mammals including humans. Infectious hematopoietic necrosis virus (IHNV), the type species of the genus Novirhabdovirus, is an important viral pathogen of salmonid fish, and is prevalent in Europe, North America and Asia. Lettuce necrotic yellow virus (LNYV), the type species of the genus Cytorhabdovirus, is an important plant virus that infects lettuce. Marburg virus (MARV), and Ebolavirus (EBOV), members of the family Filoviridae, are the two deadliest viral pathogens in humans. Mump virus (MuV), the type species of the genus Rubulavirus infects humans. Newcastle disease virus (NDV), the type species of the genus Aulavirus, infects chickens. Human respiratory syncytial virus (HRSV), the type species of the genus Pneumovirus infects humans. Citrus psorosis virus (CPsV), the type species of the genus Ophiovirus, causes diseases in citrus.

The genome of the viruses belonging to the Order Mononegavirales, families Bornaviridae, Rhabdoviridae, Filoviridae, and Paramyxoviridae contain negative sense, single-stranded, linear, non-segmented RNA, 8.9-19.0 kb in size and encodes envelope glycoprotein(s), a matrix protein, a major RNA binding protein, nucleocapsid associated protein(s), and a large polymerase protein. The 5′- and the 3′ terminal ends of the viruses contain inverse complementarity and conserved motives (Pringle 2005)

In the following section, the details of the invention are described using Human respiratory syncytial virus (HRSV) as an example. HRSV is the major cause of lower respiratory tract infections in infants and children. HRSV genome is approximately 15.2 kb in length and encodes for protein representing nonstructural protein 1 (NS1), nonstructural protein 2 (NS2), nucleocapsid protein (NC), phosphoprotein (P), matrix protein (M1), small hydrophobic protein (SH), glycoprotein (G), fusion glycoprotein (F), 22K/M2 protein and L protein. There are two antigenic sub-groups of HRSV, called A and B, that show over 95% similarity in the N protein and 53% similarity in the G protein. The F proteins on the surface of the virus cause the cell membranes on nearby cells to merge, forming syncytia, from which the name of the virus was derived.

### EXAMPLE 7

Cloning of a full-length genome of HRSV in a pCR®-XL-TOPO® vector.

The entire genome of HRSV may be amplified by RT-PCR using virus-specific primers and cloned into a pCR®-XL-TOPO® vector following manufacturer's recommendations (Invitrogen Inc., Carlsbad, Calif.). Upon cloning, the recombinant clones are verified by sequencing before taking for sub-cloning.

### EXAMPLE 8

Cloning of a T7 terminator sequence and T7 promoter sequence downstream of Gal10 promoter in the yeast expression vector, pESC-ura, and sub-cloning of HRSV genome in the yeast expression vector.

The T7 terminator sequence (5′-TAGCATAACCCCTTGGGGCCTCTAAA CGGGTCTTGAGGGGTTTTTTG-3′) (SEQ ID NO: 1) and T7 promoter sequence (5′-TAA TAC GAC TCA CTA TAG GG GAG A-3′) (SEQ ID NO: 2) are cloned downstream of Gal 10 promoter in pESC-ura vector (Stratagene, San Diego, Calif.) using unique restriction enzymes. Subsequently, the entire genome of HRSV is amplified from the plasmid clone (as described in the Example 7) by PCR and the resulting amplicon is ligated downstream of the Gal 10 promoter and T7 terminator in the pESC-ura vector by In Fusion PCR (Clonetech Corp.) generating a full-length HRSV clone under the control of Gal10 promoter with T7 terminator sequence at the upstream, and T7 promoter sequence downstream of the HRSV sequence, respectively.

Alternatively, the entire genome of HRSV with the T7 terminator sequence (5′-TAGCATAACCCCTTGGGGCCTCTAAACGGGTCTTGAGGGGTTTTTTG-3′) (SEQ ID NO: 3) upstream of the HRSV genome, and T7 promoter sequence (5′-TAATACGACTCACTATAGGGGAG A-3′) (SEQ ID NO: 4) located downstream of the viral genome can be custom synthesized and cloned into a plasmid vector, pUC57 (GenScript USA Inc., 860 Centennial Avenue, Piscataway, N.J. 08854).

Upon sequence validation, the full-length genome of HRSV along with the T7 terminator and T7 promoter sequences may be digested using unique restriction enzyme(s) and cloned into the homologous sites in the pESC-ura vector under the control of Gal 10 promoter. Another option is to amplify the full-length genome of HRSV along with the T7 terminator and T7 promoter sequences and cloned into pESC-ura by overlapping PCR. The plasmid DNA is then isolated from the recombinant clones and sequenced. The recombinant plasmid is then used to transform yeast (Saccharomyces cerevisiae strain YPH501, Stratagene, La Jolla, Calif.) using a published protocol (http://www.genomics.agilent.com/files/Manual/217451.pdf (Agilent Technologies, Santa Clara, Calif.). After transformation, the transformants are plated on SG-dropout media and grown at 30° C. for two days before taking for western blot analysis.

### EXAMPLE 9

Identification of recombinant yeast clones expressing HRSV proteins by western blot analysis.

In order to identify recombinant yeast clone expressing HRSV proteins, western blot analysis is performed using HRSV-G protein specific monoclonal antibody made in mouse (MAb-RSV-G-1, https://www.antibodies-online.com) following a published protocol (Allnutt et al., 2007). Recombinant yeast (Saccharomyces cerivisiae strain YH501; Stratagene, La Jolla, Calif.) clones containing HRSV genome are grown in autotrophic SG-ura medium containing galactose, yeast extract without amino acids, and amino acid dropout mixture (all amino acids plus adenine, no uracil) at 30° C. for 4 days. Cells were collected by centrifugation, and crude protein extracts were prepared using Y-PER yeast breaking buffer (Pierce Biotechnology, Rockford, Ill.). An aliquot of total protein is then electrophoresed in a 12% SDS-polyacrylamide gel (BioRad, Richmond, Calif.) and transferred to PVDF membrane by electroblotting. The membrane is then probed with Mouse anti-HRSV Monoclonal Antibody (Abcam Inc., Cambridge, Mass.) and detected with Goat-anti-mouse polyclonal antibody conjugated to HRP (Thermo Fisher Scientific, Rockford, Ill.). Detection is carried out using a ECL Plus Western Blotting Reagent Pack (GE Healthcare, Piscataway, N.J.).

### EXAMPLE 10

Purification of recombinant HRSV.

Recombinant HRSV is purified following essentially the same protocols as described for the purification of virus-like particles from recombinant yeast (Allnutt et al., 2007). Briefly, recombinant yeast expressing HRSV is grown in autotrophic SG-ura medium at 30° C. for 4 days. Cells were then collected by centrifugation, treated with β-glucoronidase to prepare the protoplast following a published protocol (Pannunzio et al., 2004) before storing at −20° C. Protoplasted cells are then lysed by three freeze thaw (3 cycles), and then sonicated by 60 second pulses with 20 second intervals (5 cycles). The sample is then centrifuges at 10,000 rpm for 20 minutes at 4° C. The supernatant is layered over 15-60% (w/v) sucrose gradient and centrifuged at 28,000 rpm for 3 hours at 4° C. in a swinging bucket rotor (Beckman SW20). Multiple fractions are collected throughout the gradient and western blot analysis is performed using anti-NV antibody to determining which fraction(s) contains highest level of the virus. Those fraction(s) are then diluted in diluted 1:10 volume using TN buffer (50 mM Tris and 100 mM NaCl, pH 8.0) and is centrifuged at 28,000 rpm for 2 hours at 4° C. in a swinging bucket rotor (Beckman SW20). The pellet is collected and suspended in TN buffer before using for transmission electron microscopy (TEM).

### Example 11

Characterization of HRSV purified from yeast by transmission electron microscopy, SDS PAGE, and glycosylation pattern.

Transmission electron microscopy (TEM) is performed to determine the morphology and size of the recombinant HRSV expressed in yeast following to a previously published protocol (Dykstra, 1992). An aliquot of purified HRSV produced in yeast as described in the Example 10 is boiled in a denaturing buffer containing 0.5% sodium dodecyl sulfate (SDS) and 1% β-mercaptoethanol for 10 minutes, and subjected to SDS polyacrylamide gel electrophoresis (SDS-PAGE) on 12% gels. The proteins from the gel are transferred to nylon membrane and the membrane is probed with anti- HRSV antibody to demonstrate the reactivity of the purified HRSV to homologous antibody.

In order to determine the glycosylation pattern of the yeast expressed HRSV, purified virions are pre-treated with 10 U endoglycosidase (Endo) H (New England Biolabs, Beverly, Mass.) per microgram of protein at 37° C. for 3 hours in a buffer containing 50 mmol/L sodium citrate at pH 5.5 prior to denaturation and separation using SDS-PAGE to remove any glycosylation from the viral coat proteins. Untreated purified wild-type HRSV sample serves as a control. The comparison of HRSV peptide profiles upon treatment of endoglycosidase vs. control sample is used to determine the extent of glycosylation of the yeast expressed HRSV relative to that of the native HRSV. Comparison of deglycosylated control HRSV proteins with deglycosylated HRSV proteins produced by the instant invention using SDS-PAGE establishes that the HRSV proteins produced using the instant invention are processed accurately (i.e., to the same molecular weight as in the native HRSV).

### EXAMPLE 12

Establishing the infectivity of HRSV isolated from Example 7 by in vitro assay.

In order to determine the infectivity of the recombinant HRSV purified from yeast cells, mammalian cells such as HEP-2 and HeLa are inoculated with the purified virus and the development of cytopathic effect is monitored upon viral inoculation (http://virology-online.com/viruses/RSV6.htm). In addition, HRSV load in the inoculated cells, as infection progresses, may be measure by real-time quantitative RT-PCR following a published protocol (Brooks 2005).

### EXAMPLE 13

Establishing the infectivity of yeast expressed HRSV in animal model and determining the anitgencity of inactivated HRSV.

The infectivity of the yeast expressed HRSV is demonstrated in animal model using cotton rat or mice (Byrd and Prince, 1997). In addition formalin inactivated yeast expressed HRSV can be injected into cotton rat or mice and ability of the inactivated virus in eliciting anti-HRSV antibody response is measured to determine the efficacy of the HRSV vaccine.

[symbol]

### REFERENCES

The contents of all references cited herein are incorporated by reference herein for all purposes:
Allnutt, F. C., Rowe, C., Bowers, R. M., Vakharia, V., LaPatra, S. E. and Dhar, A. K. 2007. Antigenicity of infectious pancreatic necrosis virus VP2 sub-viral particle expressed in yeast. Vaccine 25: 4880-4888.
Brooks, H. A., Gersberg, R. M., and Dhar, A. K. 2005. Detection and quantification of Hepatitis A virus in seawater via real-time RT-PCR, Journal of Virological Methods 127: 109-118.
Byrd, L. G. and Prince, G. A. 1997. Animal models of respiratory syncytial virus infection. Clin Infect Dis. 25:1363-8.
Dykstra, M. J., 1992. Specimen preparation for transmission electron microscopy, in: Dykstra M J (ed.), Biological Electron Microscopy. New York, London: Plenum Press, pp. 5-78.
Goodgame R (2006)). “Norovirus gastroenteritis”. Curr Gastroenterol Rep 8 (5): 401-8).
Guix, S., Asanaka, M., Katayama, K., Crawford, S. E., Neill, F. H., Atmar, R. L. and Estes, M. K. 2007. Norwalk virus RNA is infectious in mammalian cells. J. Virol. 81: 12238-12248.
Jiang, X., M. Wang, K. Wang, and M. K. Estes. 1993. Sequence and genomic organization of Norwalk virus. Virology 195:51-61.
Pannunzio, V. G., Burgos, H. I., Alonso, M., Ramos, E. H., Mattoon, J. R., Stella, C. A. 2004. Yeast Plasmids with the Least Trouble. Promega Notes 87: 27-28.
Pringle, C. R. 2005 Order: Mononegavirales, In: Virus Taxonomy, Eight Report of the International Committee on Taxonomy of Viruses. (Eds.) C. M. Faquet, M. A. Mayo, J. Maniloff, U. Desselberger, L. A. Ball, International Union of Microbiological Societies, pp 609-614.
Tan, M. and Jiang, X. 2010. Norovirus Gastroenteritis, Carbohydrate Receptors, and Animal Models. PLoS Pathogen, Aug, Vol 6, Issue 8.

1. A composition comprising an infectious RNA virus that codes for the entire functional genome of a single-stranded positive-sense RNA virus or negative-sense RNA virus expressed in yeast that will not otherwise replicate in yeast and wherein the expression of the DNA fragment is under the control of a single or more than one promoter.

2. The composition of claim 1, wherein the promoter is functional in yeast.

3. The composition of claim 1, wherein the promoter is a pantropic promoter.

4. The composition of claim 1, wherein the promoter is a cell-type specific promoter.

5. The composition of claim 1, wherein the positive-sense single stranded RNA virus is selected from the group consisting of a Norwalk virus (NV); hepatitis C virus (HCV);dengue virus (DENV); and West Nile virus (WNV) and the negative-sense RNA virus is selected from the group consisting is a respiratory syncytial virus (RSV) and viral hemorrhagic septicemia virus (VHSV).

6. A composition comprising an infectious RNA virus that codes for the entire functional genome (complementary DNA cDNA) of a single-stranded positive-sense RNA virus or a single-stranded negative-sense RNA virus expressed in yeast that will not otherwise replicate in yeast and wherein the expression of the DNA fragment is under the control of a single or more than one promoter and where the said composition also contains a second fragment of complimentary DNA that codes for a sub-genomic component of the same single stranded positive-sense or negative-sense RNA virus representing the structural genes, and wherein the expression of the sub-genomic component representing the structural genes is under control of a second promoter.

7. The composition of claim 6, wherein the promoters driving the entire functional genome of the virus and the sub-genomic fragment representing the structural genes are functional in yeast.

8. The composition of claim 6, wherein the promoters driving the entire functional genome of the virus and the sub-genomic fragment representing the structural genes are pantropic promoters.

9. The composition of claim 6, wherein the promoters driving the entire functional genome of the virus and the sub-genomic fragment representing the structural genes are cell-type specific promoters.

10.-18. (canceled)

19. A method for the production of a vaccine comprising a fully-assembled virus containing the entire genome of the vaccine virus or a truncated genome of the vaccine virus (attenuated virus) that has been purified to homogeneity and/or inactivated prior to vaccination.

20. The method of claim 19, further comprising producing a fully-assembled virus containing a sub-genomic fragment of the vaccine virus that has been purified to homogeneity and inactivated prior to vaccination.

21.-22. (canceled)

23. The method of claim 19 further comprising a fully-assembled virus containing a sub-genomic fragment of the vaccine virus that has been inactivated prior to vaccination.

24.-25. (canceled)


SEQUENCE LISTING (4 sequences)

SEQ ID NO: 1 - DNA, 47 bp, Artificial Sequence
  Synthetic construct
tagcataaccccttggggcctctaaacgggtcttgaggggttttttg

SEQ ID NO: 2 - DNA, 24 bp, Artificial Sequence
  Synthetic Construct
taatacgactcactataggggaga

SEQ ID NO: 3 - DNA, 47 bp, Artificial Sequence; identical to SEQ ID NO: 1
  Synthetic construct

SEQ ID NO: 4 - DNA, 24 bp, Artificial Sequence
  Synthetic construct
taatacgactcactataggggaga